(19) United States
(12) Patent Application Publication
(10) Pub. No.: US 2026/0159489 A1
(43) Pub. Date: Jun. 11, 2026

(54) Deuterium-Enriched 6-Carboxy-2-(3,5-Dichlorophenyl)Benzoxazoles for Treatment of Transthyretin Amyloidosis Cardiomyopathy (ATTR-CM), Polyneuropathy (ATTR-PN), and Related Diseases

(71) Applicant: Daljit Singh Dhanoa, Del Mar, CA (US)

(72) Inventor: Daljit Singh Dhanoa, Del Mar, CA (US)

(21) Appl. No.: 18/973,808

(22) Filed: Dec. 9, 2024

(51) Int. Cl. C07D 263/57; C07B 59/00

(52) U.S. Cl. CPC C07D 263/57; C07B 59/002

(57) ABSTRACT

The present invention is concerned with novel deuterium-enriched compounds of the general chemical structural formula I, and pharmaceutically acceptable salts, compositions, and methods of use thereof,

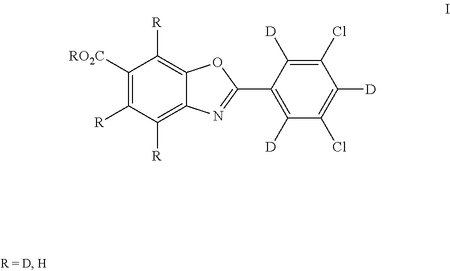

wherein,
R is independently deuterium (D), hydrogen (H) atom. Compounds of general chemical structure I are stabilizers of the Transthyretin (TTR) protein and are useful in the treatment of Transthyretin Amyloidosis (ATTR) and related diseases.

### PRIORITY CLAIM

This application is a continuation-in-part of U.S. Provisional Patent Application No. 63/608,333, which is incorporated by reference in its entirety for all purposes.

### FIELD OF THE INVENTION

This invention relates to disclosing novel deuterium-enriched 6-Carboxy-2-(3,5-Dichlorophenyl)benzoxazole compounds, pharmaceutical salts, compositions and methods of use thereof for stabilizing a protein namely transthyretin (TTR), inhibiting transthyretin misfolding, fibrils formation, aggregation and deposition of amyloid fibrils into various organs and tissues including heart, lung, kidney, liver, brain, and nervous system, and thereby treating transthyretin-mediated diseases, including transthyretin amyloidosis (ATTR) and other related diseases associated thereto.

### BACKGROUND OF THE INVENTION

Transthyretin (TTR) is a 55 kDa homotetrameric protein abundantly present in serum and cerebrospinal fluid (CSF). Transthyretin (TTR) is a tetrameric protein produced in the liver that serves as a plasma transport protein carrying thyroxine and retinol. In patients with transthyretin amyloidosis (ATTR), unstable tetramer structure of thyretin protein (TTR) may dissociate due to genetic mutation (e.g. V122I mutation) or age and misfold into amyloid fibrils. These fibrils can then deposit in various tissues and/or organs throughout the body, leading to a constellation of symptoms. Transthyretin amyloidosis (ATTR) has two types, hereditary or wild type. Hereditary transthyretin amyloidosis (hATTR) is inherited in an autosomal dominant manner with variable penetrance. Mutations in the TTR gene lead to the formation of abnormal proteins. Wild-type ATTR (ATTRwt) develops with age and is acquired when normal TTR tetramers destabilize and become amyloidogenic.

ATTRwr classically is present with cardiomyopathy, carpal tunnel syndrome, and radiculopathy in older patients, usually above the age of 60 years. hATTR amyloidosis is seen in younger and older patients, resulting in peripheral cardiomyopathy, neuropathy, or a combination of both cardiomyopathy and neuropathy. The phenotypes vary depending on the mutation.

Post-secretion amyloidogenesis of plasma TTR requiring rate limiting tetramer dissociation, monomer misfolding and misassembly putatively causes familial amyloid cardiomyopathy and the familial amyloid polyneuropathies, and senile systemic amyloidosis.

The TTR amyloid diseases include but are not limited to familial transthyretin amyloidosis cardiomyopathy (ATTR-CM) or familial transthyretin amyloidosis polyneuropathy (ATTR-PN), leptomeningeal amyloids, oculoleptomengial amyloidosis, senile systemic amyloidosis, vitreous amyloidosis, and CNS amyloidosis. Amyloid diseases also include but are not restricted to ocular amyloidosis, gastrointestinal amyloidoses, neuropathic amyloidoses, non-neuropathic amyloidoses, nephropathy, non-hereditary amyloidoses, reactive/secondary amyloidoses, cerebral amyloidoses. Alzheimer's disease, spongiform encephalopathy (i.e. Creutzfeldt Jakob disease, GSS, fatal familial insomnia), frontotemporal dementia, Parkinson's disease, amyotrophic lateral sclerosis (ALS). Down Syndrome, multiple sclerosis, polyneuropathy, Guillain-Barré syndrome, macular degeneration, vitreous opacities, glaucoma, type II diabetes and medullary carcinoma of the thyroid.

General and non-invasive interventions to ameliorate TTR amyloidosis (ATTR) preferably by orally active drugs are in great need to treat ATTR and related diseases.

TTR is a 55 kDa tetramer composed of 127 amino-acid subunits exhibiting an unusually high β-sheet content. It possesses a conserved structural prototype of four identical monomeric subunits that form a central channel or binding site [Gasperini R. J., et al. Mechanisms of transthyretin aggregation and toxicity. Subcell. Biochem. 2012, 65, 211; Kelly J. W., et al. Transthyretin quaternary and tertiary structural changes facilitate misassembly into amyloid. Adv. Protein. Chem. 1997, 50, 161].

Structural changes in amyloidogenic TTR have been difficult to identify through X-ray crystallography; however, novel studies with nuclear magnetic resonance spectroscopy have revealed small chemical shifts to the backbone structure of mutated and WT-TTR [Leach B. I., Zhang X., Kelly J. W., Dyson H. J., Wright P. E. NMR Measurements reveal the structural basis of transthyretin destabilization by pathogenic Mutations. Biochemistry. 2018, 57, 4421]. These changes are significant to the TTR ground state and the most significant changes were found in proximity to the site of the mutation. These subtle structural differences are just one consequence of the mutation, and different TTR variants might experience diverse structural deformation pathways, resulting in atypical responses to the cellular conditions or proteolytic stress.

ATTR-CM and ATTR-PN are rare, progressive and under-diagnosed ATTR-mediated potentially fatal diseases. Effective therapeutics are unavailable for the treatment or prevention of transthyretin amyloidosis (ATTR) and ATTR-mediated diseases. The present invention discloses novel deuterium-containing compounds that stabilize TTR dissociation and prevent TTR aggregation and provide therapeutics for the prevention and treatment of transthyretin amyloidosis (ATTR) and ATTR-mediated cardiomyopathy (ATTR-CM) and related ATTR-mediated diseases.

### SUMMARY OF THE INVENTION

The present invention is concerned with novel deuterium-enriched compounds of the general chemical structural formula I, and pharmaceutically acceptable salts, compositions, and methods of use thereof,

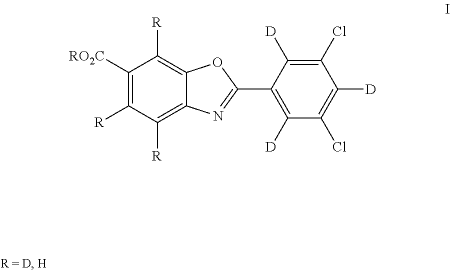

wherein,
R is independently deuterium (D).

Compounds of formula I are stabilizers of the tetrameric protein, Transthyretin (TTR), thereby these deuterium-enriched compounds of formula I can prevent TTR dissociation and its subsequent aggregation, and deposition in organs and tissues, including heart, lungs, kidney, liver, brain, nervous system, and thereby treat Transthyretin Amyloidosis (ATTR) and ATTR-mediated diseases.

The compounds of general chemical structural formula I, and their pharmaceutical salts are Transthyretin (TTR) stabilizers, and are useful for the treatment of diseases mediated by TTR including Transthyretin Amyloidosis (ATTR) and related diseases.

Transthyretin Amyloidosis (ATTR) is a systemic, progressive, debilitating and life-threatening disease with a constellation of clinical manifestations. Transthyretin Amyloidosis (ATTR) is caused by formation, aggregation, and deposition of amyloid fibrils from destabilization of the tetrameric TTR in different organs and tissues including heart and nervous system and other organs including kidneys, resulting in end organ damage and their dysfunction. Transthyretin (TTR) amyloidosis is an under diagnosed disease that is characterized by misfolding of TTR and aggregation as amyloid fibrils, predominantly leading to transthyretin amyloidosis cardiomyopathy (ATTR-CM) or transthyretin amyloidosis polyneuropathy (ATTR-PN) depending on the particular TR mutation. Transthyretin amyloid cardiomyopathy (ATTR-CM) can also occur as an age-related disease caused by misfolding of wild-type TTR.

Deuterium-enriched Compounds of chemical structural formula 1 disclosed in the present invention are oral transthyretin (TTR) stabilizers for the treatment of transthyretin amyloidosis (ATTR) diseases including but not limited to transthyretin amyloidosis cardiomyopathy (ATTR-CM) and transthyretin amyloidosis polyneuropathy (ATTR-PN). Significant unmet medical need exists for prevention and/or treatment of ATTR-mediated diseases including ATTR-CM, ATTR-PN, and other diseases.

In this invention, we disclose the design and synthesis of highly potent and selective novel deuterium-enriched 6-Carboxy-2-(3,5-dichlorophenyl)benzoxazole compounds as Transthyrin (TTR) stabilizers that can treat transthyretin amyloidosis (ATTR) diseases including but not limited to transthyretin amyloidosis cardiomyopathy (ATTR-CM) and transthyretin amyloidosis polyneuropathy (ATTR-PN).

These novel deuterium-enriched compounds of chemical structural formula I and pharmaceuticals salts thereof can also treat leptomeningeal amyloids, oculoleptomengial amyloidosis, senile systemic amyloidosis, vitreous amyloidosis, and CNS amyloidosis. Amyloid diseases also include but are not restricted to ocular amyloidosis, gastrointestinal amyloidoses, neuropathic amyloidoses, non-neuropathic amyloidoses, nephropathy, non-hereditary amyloidoses, reactive/secondary amyloidoses, cerebral amyloidoses. Alzheimer's disease, spongiform encephalopathy (i.e. Creutzfeldt Jakob disease, GSS, fatal familial insomnia), frontotemporal dementia, Parkinson's disease, amyotrophic lateral sclerosis (ALS). Down Syndrome, multiple sclerosis, polyneuropathy, Guillain-Barré syndrome, macular degeneration, vitreous opacities, glaucoma, type II diabetes and medullary carcinoma of the thyroid.

This invention further constitute a method for stabilizing tetrameric structure of the transthyretin (TTR) protein preventing from its dissociation into its monomeric forms leading to protein misfolding and formation, aggregation, and deposition of amyloid fibrils in organs and tissues, thereby causing transthyretin amyloidosis (ATTR) in mammal, including humans, which comprises administering to a mammal in need of such treatment an effective amount of a compound of structural Formula I and pharmaceuticals salts thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The present invention relates to novel deuterium-enriched compounds of general formula I, and pharmaceutically acceptable salts, compositions, and methods of use thereof,

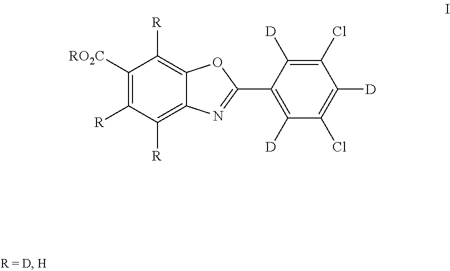

wherein,
R is independently deuterium (D).

The compounds of formula I and pharmaceutically acceptable salts and combinations thereof, have activity for stabilizing transthyretin and are therefore useful in the treatment of diseases involving tetrameric transthyretin degradation, aggregation and accumulation of dissociated monomers into fibril deposits in various tissues and organs and thereby leading to their dysfunction and end-organ damage including heart, kidneys, brain and other organs and tissues.

Transthyretin (TTR) is an abundant protein in serum and cermbrospinal fluid (CSF). Transthyretin (TTR) is a tetramer protein produced in the liver that serves as a plasma transport protein carrying thyroxine and retinol. TTR is a 55 kDa tetramer composed of 127 amino-acid subunits exhibiting an unusually high β-sheet content. It possesses a conserved structural prototype of four identical monomeric subunits that form a central channel or binding site [Gasperini R. J., et al. Mechanisms of transthyretin aggregation and toxicity. Subcell. Biochem. 2012, 65, 211-224; Kelly J. W., et al. Transthyretin quaternary and tertiary structural changes facilitate misassembly into amyloid. Adv. Protein. Chem. 1997, 50, 161-181].

Pathogenic mutations accelerate TTR aggregation by thermodynamic or kinetic destabilization of the protein [Hammerstrom. P., et al. Proc. Natd. Acad. Sci. U.S.A, 2002, 99, 16427-164321; Hurshman, A. R., et al. Biochemistry 2008, 47, 6969-694]. Familial amyloid cardiomyopathy (FAC) and Familial amyloid polyneuropathy (FAP) are the most prevalent ATTR diseases affecting the heart and peripheral nervous system, respectively [Jacobson, D. R., et al. N. Eng. J. Med. 1997, 336.466-473; Andrade, C. A., Brain. 1952, 75, 408-427; Saraiva. M. J., J. Clin. Invest. 1984, 74, 104-119]. Rare mutations are involved in the central nervous system [Goren, H., et al. Brain 1980, 103, 473-495; Sekijima, Y., et al. Lab Invest. 2003, 83, 409-417]. FAC and FAP are also called transthyretin amyloidosis cardiomyopathy (ATTR-CM) and transthyretin amyloidosis polyneuropathy (ATTR-PN) respectively.

In patients with transthyretin amyloidosis (ATTR), unstable tetrameric structure of thyretin protein (TTR) may dissociate due to genetic mutation (e.g. V122I mutation) or age, and misfolding into amyloid fibrils. These fibrils can then deposit in various tissues and/or organs throughout the body, leading to a constellation of symptoms. Transthyretin Amyloidosis (ATTR) is caused by formation, aggregation, and deposition of amyloid fibrils from destabilization of the tetrameric TTR in different organs and tissues including heart and nervous system and other organs including kidneys, resulting in end organ damage and their dysfunction.

Transthyretin amyloidosis (ATTR) has two types, hereditary or wild type. Hereditary transthyretin amyloidosis (hATTR) is inherited in an autosomal dominant manner with variable penetrance. Mutations in the TTR gene lead to the formation of abnormal proteins. Wild-type ATTR (ATTRwt) develops with age and is acquired when normal TTR tetramers destabilize and become amyloidogenic. ATTR_{WT} classically is present with cardiomyopathy, carpal tunnel syndrome, and radiculopathy in older patients, usually above the age of 60 years. hATTR amyloidosis is seen in younger (early-onset) and older patients (late-onset), resulting in peripheral neuropathy, cardiomyopathy, or a mixed combination. The phenotypes vary depending on the mutation.

General and non-invasive interventions to ameliorate TTR amyloidosis (ATTR) preferably by orally active drugs are of great interest and tremendous need for addressing unmet need to treat ATTR and related diseases.

Traditional drugs such as beta-blockers, angiotensin-converting enzyme inhibitors (ACEI), or angiotensin receptor antagonists/blockers (ARB) for symptomatic treatment, cannot slow, halt, or reverse the underlying cause of ATTR-CM.

The novel deuterium-enriched compounds disclosed in this invention are effective kinetic stablizers of the tetramer protein transthyretin (TTR) that inhibit dissociation of the non-amyloidogenic native state of TTR protein. The stabilization of the native state of TTR by compounds of formula I also can treat neurodegerative diseases since misfolded oligomers have been reported to cause neurodegeneration.

Deuterium-enriched Compounds of formula 1 disclosed in the present invention are oral transthyretin (TTR) stabilizers for the treatment of transthyretin amyloidosis (ATTR) diseases including but not limited to transthyretin amyloidosis cardiomyopathy (ATTR-CM) and transthyretin amyloidosis polyneuropathy (ATTR-PN). Significant unmet medical need exists for prevention and/or treatment of ATTR-mediated diseases including ATTR-CM, ATTR-PN, and other diseases.

In this invention, we disclose the design and synthesis of highly potent and selective novel deuterium-enriched 6-Carboxy-2-(3,5-DichloroPhenyl)benzoxazole compounds as Transthyrin (TTR) stabilizers that can treat transthyretin amyloidosis (ATR) diseases including but not limited to transthyretin amyloidosis cardiomyopathy (ATTR-CM) and transthyretin amyloidosis polyneuropathy (ATTR-PN).

These novel deuterium-enriched compounds of formula I and pharmaceuticals salts thereof can also treat leptomeningeal amyloids, oculoleptomengial amyloidosis, senile systemic amyloidosis, vitreous amyloidosis, and CNS amyloidosis. Amyloid diseases also include but are not restricted to ocular amyloidosis, gastrointestinal amyloidoses, neuropathic amyloidoses, non-neuropathic amyloidoses, nephropathy, non-hereditary amyloidoses, reactive/secondary amyloidoses, cerebral amyloidoses, Alzheimer's disease, spongiform encephalopathy (i.e. Creutzfeldt Jakob disease, GSS, fatal familial insomnia), frontotemporal dementia, Parkinson's disease, amyotrophic lateral sclerosis (ALS), Down Syndrome, multiple sclerosis, polyneuropathy, Guillain-Barre syndrome, macular degeneration, vitreous opacities, glaucoma, type II diabetes and medullary carcinoma of the thyroid.

Deuterium (D or ²H) is a stable non-radioactive isotope of hydrogen (H) and has an atomic weight of 2.0144. Hydrogen occurs naturally as a mixture of the isotopes ¹H, D (²H or deuterium), and T (³H or tritium) and the natural abundance of deuterium is 0-015%. One of ordinary skill in the art recognizes that in all compounds containing H atom, H actually represents a mixture of H and D, with about 0-015% of D. So therefore, compounds with a level of D (deuterium) that has been enriched to be greater than its natural abundance of 0.015%, should be considered unnatural and as a result novel as compared to their corresponding non-enriched counterparts.

The carbon-hydrogen bonds contain a naturally occurring distribution of hydrogen isotopes, namely ¹H or protium (about 99.9844%), ²H or deuterium (D) (about 0.0156%), and ³H or tritium (in the range between about 0.5 and 67 tritium atoms per 1018 protium atoms). Higher levels of deuterium incorporation produce a detectable Kinetic Isotope Effect [Werstiuk, N. H.; Dhanoa, D. S.; Timmins, G. Can J. Chem. 1979, 57, 2885; Werstiuk, N. H.; Dhanoa, D. S.; Timmins, G. Can J. Chem. 1983, 61, 2403], that could improve the pharmacokinetic, pharmacologic and/or toxicologic parameters of compounds of formula I in comparison to compounds having naturally occurring levels of deuterium and their corresponding hydrogen (protium) analogs.

Suitable modifications of certain carbon-hydrogen (C—H) bonds into carbon-deuterium (C-D) bonds can generate novel substituted compounds of structural formula I with unexpected and non-obvious improvements of pharmacological, pharmacokinetic and toxicological properties in comparison to the non-isotopically enriched compounds. This invention relies on the judicious and successful application of chemical kinetics to drug design. Deuterium incorporation levels in the compounds of the invention are significantly higher than the naturally-occurring levels and are sufficient to induce at least one substantial improvement as described herein.

“Deuterium enrichment” refers to the percentage of incorporation of deuterium at a given site on the molecule instead of a hydrogen atom. For example, deuterium enrichment of 1% means that in 1% of molecules in a given sample a particular site is occupied by deuterium. Because the naturally occurring distribution of deuterium is about 0.0156%, deuterium enrichment in compounds synthesized using non-enriched starting materials is about 0.0156%.

It can be a significant synthetic challenge to produce 100% deuterium at a specific site of a compound. High levels of deuterium content in a compound can be produced either by Hydrogen—Deuterium (H-D) exchange or by synthesizing the compound for specific deuteration. The H-D exchange is readily achieved in case of H atoms attached to heteroatoms for example in cases of carboxylic acids (COOH), sulfonamides (SO₂NH₂, CONHSO₂-aryl, CONHSO₂-alkyl), alcohols (OH), basic amines (NH₂), etc. However, these incorporated deuterium (D) attached to heteroatoms (O, N, S) readily revert back to hydrogen (H) upon exposure to water or any acidic compounds containing H atoms. The preferred deuterium containing compounds contain deuterium (D) directly attached to carbon atoms of the structure of the compounds of this present invention.

In some embodiments, the deuterium enrichment in the compounds of the present invention is greater than 4%, 5%, 6%, 7%, 8%, 9% or 10%. In other embodiments, the deuterium enrichment in the compounds of the present invention is greater than 20%. In further embodiments, the deuterium enrichment in the compounds of the present invention is greater than 50%. In some embodiments, the deuterium enrichment in the compounds of the present invention is greater than 70%. In some embodiments, the deuterium enrichment in the compounds of the present invention is greater than 90%.

The present invention relates to novel deuterium-enriched 6-Carboxy-2-(3,5-dichlorophenyl)benzoxazole compounds of general chemical structural formula I, and pharmaceutically acceptable salts, compositions, and methods of use thereof,

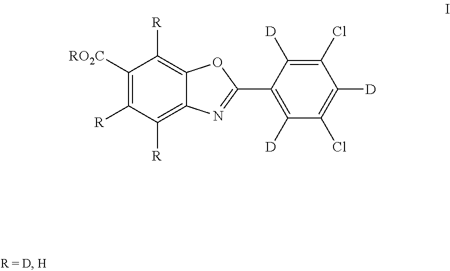

wherein,
R is independently deuterium (D), H.

Wherein D is deuterium atom present in the compounds of formula I and about 1%-100% enrichment of deuterium is incorporated.

In some embodiments, a compound of the invention is of the formula of I,

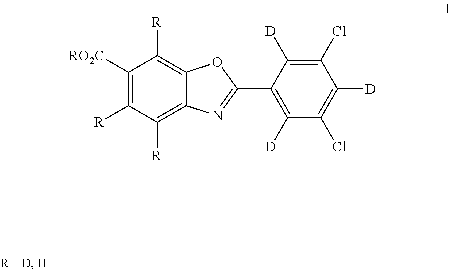

and pharmaceutically acceptable salts, compositions, and methods of use thereof,
wherein,
R is independently deuterium (D), H.

In another embodiments, a compound of the present invention is a deuterium-enriched compound, and pharmaceutically acceptable salts, compositions, and methods of use thereof, of name and formula:
(a) 2-(3,5-dichlorophenyl-2,4,6-d₃)-1,3-benzoxazole-6-carboxylic acid-d,

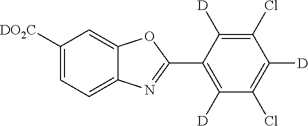

(b) 2-(3,5-dichlorophenyl-2,4,6-d₃)-1,3-benzoxazole-6-carboxylic acid,

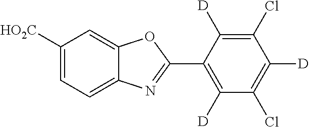 [SMILES: [2H]c1c(Cl)c([2H])c(-c2nc3ccc(C(=O)O)cc3o2)c([2H])c1Cl]

(c) 2-(3,5-dichlorophenyl-2,4,6-d₃)-1,3-benzoxazole-6-carboxylic-7-d acid-d,

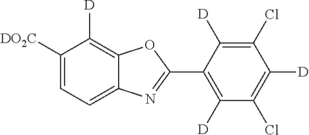

(d) 2-(3,5-dichlorophenyl-2,4,6-d₃)-1,3-benzoxazole-6-carboxylic-7-d acid,

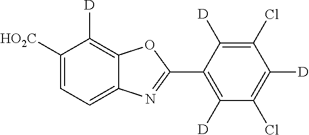 [SMILES: [2H]c1c(Cl)c([2H])c(-c2nc3ccc(C(=O)O)c([2H])c3o2)c([2H])c1Cl]

(e) 2-(3,5-dichlorophenyl-2,4,6-d₃)-1,3-benzoxazole-6-carboxylic-4,7-d₂ acid-d,

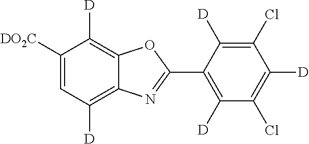

(f) 2-(3,5-dichlorophenyl-2,4,6-d₃)-1,3-benzoxazole-6-carboxylic-4,7-d₂ acid,

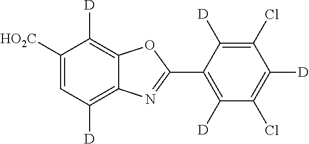 [SMILES: [2H]c1c(Cl)c([2H])c(-c2nc3c([2H])cc(C(=O)O)c([2H])c3o2)c([2H])c1Cl]

(g) 2-(3,5-dichlorophenyl-2,4,6-d₃)-1,3-benzoxazole-6-carboxylic-4,5,7-d₃ acid-d,

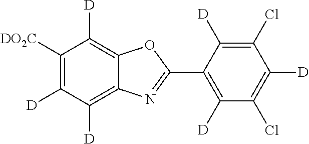

(h) 2-(3,5-dichlorophenyl-2,4,6-d₃)-1,3-benzoxazole-6-carboxylic-4,5,7-d₃ acid,

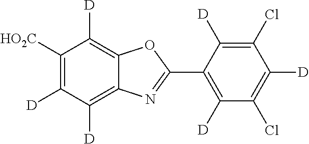 [SMILES: [2H]c1c(Cl)c([2H])c(-c2nc3c([2H])c([2H])c(C(=O)O)c([2H])c3o2)c([2H])c1Cl]

(j) 2-(3,5-dichlorophenyl-2,4,6-d₃)-1,3-benzoxazole-6-carboxylic-4-d acid-d,

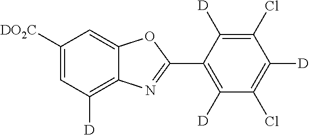

(k) 2-(3,5-dichlorophenyl-2,4,6-d₃)-1,3-benzoxazole-6-carboxylic-4-d acid,

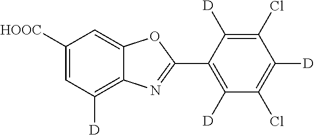 [SMILES: [2H]c1c(Cl)c([2H])c(-c2nc3c([2H])cc(C(=O)O)cc3o2)c([2H])c1Cl]

(l) 2-(3,5-dichlorophenyl-2,4,6-d₃)-1,3-benzoxazole-6-carboxylic-5,7-d₂ acid-d,

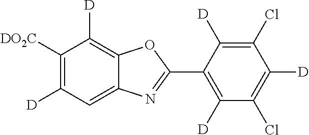

(m) 2-(3,5-dichlorophenyl-2,4,6-d₃)-1,3-benzoxazole-6-carboxylic-5,7-d₂ acid,

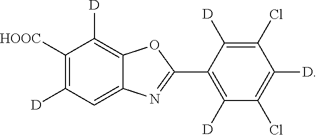

In another embodiment, a pharmaceutical composition comprising a compound of the invention of chemical structural formula I in an amount effective for the treatment of a disease selected from transthyretin amyloidosis cardiomyopathy (ATTR-CM) and transthyretin amyloidosis polyneuropathy (ATTR-PN), leptomeningeal amyloids, oculoleptomengial amyloidosis, senile systemic amyloidosis, vitreous amyloidosis, and CNS amyloidosis, ocular amyloidosis, gastrointestinal amyloidosis, neuropathic amyloidosis, non-neuropathic amyloidosis, nephropathy, non-hereditary amyloidosis, reactive/secondary amyloidosis, cerebral amyloidosis, Alzheimer's disease, spongiform encephalopathy (i.e. Creutzfeldt Jakob disease, GSS, fatal familial insomnia), frontotemporal dementia, Parkinson's disease, amyotrophic lateral sclerosis (AILS), Down Syndrome, multiple sclerosis, polyneuropathy, Guillain-Barré syndrome, macular degeneration, vitreous opacities, glaucoma, type II diabetes and medullary carcinoma of the thyroid.

In another embodiment, a method of treating a disease selected from transthyretin amyloidosis cardiomyopathy (ATTR-CM) and transthyretin amyloidosis polyneuropathy (ATTR-PN), leptomeningeal amyloids, oculoleptomengial amyloidosis, senile systemic amyloidosis, vitreous amyloidosis, and CNS amyloidosis, ocular amyloidosis, gastrointestinal amyloidosis, neuropathic amyloidosis, non-neuropathic amyloidosis, nephropathy, non-hereditary amyloidosis, reactive/secondary amyloidosis, cerebral amyloidosis. Alzheimer's disease, spongiform encephalopathy (i.e. Creutzfeldt Jakob disease, GSS, fatal familial insomnia), frontotemporal dementia, Parkinson's disease, amyotrophic lateral sclerosis (ALS), Down Syndrome, multiple sclerosis, polyneuropathy, Guillain-Barré syndrome, macular degeneration, vitreous opacities, glaucoma, type 11 diabetes and medullary carcinoma of the thyroid, comprising administering a pharmaceutically effective amount of a pharmaceutical composition of compound of formula I.

In another embodiment, the compounds of the invention described herein may also be administered in combination with other treatments for diseases caused by TTR amyloid. Treatments for diseases caused by TTR amyloid include heart transplant for TTR cardiomyopathy, liver transplant, other kinetic stabilizers of TTR. RNA knock-down and/or RNA interference methods and the like. The compound described herein the present invention of formula I may be administered before, after, or during another treatment or medical procedure including heat transplant for TTR cardiomyopathy, liver transplant, other stabilizer molecules, entities, for diseases caused by TTR amyloid.

In some embodiments, the compounds and compositions of the present invention, disclosed herein may be administered as the only therapeutic agent or in combination with other active ingredients. The compounds and compositions may be administered in combination with other compounds to treat amyloidosis and amyloid disorders, including but not limited to compounds, for example, that bind to and stabilize TTR and/or compounds that target TTR RNA and/or compounds that modulate the expression of the TTR protein and/or compounds that modulate the transcription of the TTR gene and/or compounds, which may modulate intra or extracellular protein homeostasis and/or protein stability and/or protein aggregation and/or protein folding.

Further active ingredients for combination treatments and/or therapies include but are not limited to compounds for the disease modifying or symptomatic treatment of amyloidosis, including but not limited to familial amyloid polyneuropathy, familial amyloid cardiomyopathy, cerebral amyloidosis, leptomeningeal amyloids, oculoleptomengial amyloidosis, senile systemic amyloidosis, vitreous amyloidosis, ocular amyloidosis, gastrointestinal amyloidosis, neuropathic amyloidosis, non-neuropathic amyloidosis, nephropathy, non-hereditary amyloidosis, reactive/secondary amyloidosis, Alzheimer's disease, spongiform encephalopathy (i.e. Creutzfeldt Jakob disease. GSS, fatal familial insomnia), Guillain-Barré syndrome, frontotemporal dementia, multiple sclerosis, polyneuropathy, Parkinson's disease, amyotrophic lateral sclerosis (ALS), Down Syndrome, macular degeneration, vitreous opacities, glaucoma, type II diabetes and medullary carcinoma of the thyroid.

The reaction scheme conceptualized and used for the synthesis of compounds and intermediates of this invention are general. It will be understood by those skilled in the art of organic synthesis that one or more functional groups present in a given compound of the invention may render the molecule incompatible with a particular synthetic sequence. In such a case an alternative synthetic route, an altered order of steps or a strategy of protection and deprotection may be employed. The reactions are performed in a solvent appropriate to the reagents and materials employed and suitable for the transformation being effected. It is understood by those skilled in the art of organic synthesis that the functionality present on the reactants and reagents being employed should be consistent with the chemical transformations being conducted. Depending upon the reactions and techniques to be used optimal yields may require changing the order of synthetic steps or use of protecting groups followed by deprotection. In all cases the particular reaction conditions, including reagents, solvent, temperature and time, should be chosen so that they are consistent with the nature of the functionality present in the molecule.

The compounds useful in the novel method treatment of this invention may form salts with various inorganic and organic acids and bases, which are also within the scope of the invention. Such salts include alkali metal salts like sodium and potassium salts, ammonium salts, alkaline earth metal salts such as calcium and magnesium salts, salts with organic bases for example dicyclohexylamine salts, N-methyl-D-glucamine salts, salts with amino acids e.g., arginine, lysine, etc. In addition, salts with organic and inorganic acids may be prepared; e.g., HCl, HBr, H₂SO₄, H₃PO₄, methanesulfonic, toluenesulfonic, maleic, fumaric, camphorsulfonic acid.

The salts can be formed by conventional means, such as by reacting the free acid or free base forms of the product with one or more equivalents of the appropriate base or acid in a solvent or medium in which the salt is insoluble, or in a solvent such as water, which is then removed in vacuo or by freeze-drying or by exchanging the cations of an existing salt for another cation on a suitable ion exchange resin.}

The compounds useful in the novel method treatment of this invention form solvates with various solvents, which are also within the scope of the invention. Such solvates include methyl tert-butyl ether solvates, diethyl ether or others.

It will be appreciated that the compounds of general Formula I in this invention may be derivatized at functional groups to provide prodrug derivatives, which are capable of conversion back to the parent compounds in vivo. The concept of prodrug administration has been extensively reviewed (e.g. A. A. Sinkula in Annual Reports in Medicinal Chemistry, Vol 10, R. V. Heinzelmann, ED., Academic Press, New York, London, 1975, Ch 13, pp 306; H. Ferres, Drugs of Today, 1983, Vol 19, 499; and J. Med. Chem., 1975, 18, 172). Examples of such prodrugs include the physiologically acceptable and metabolically labile ester derivative, such as lower alkyl (e.g. methyl or ethyl esters), aryl (e.g. 5-indanyl esters), alkenyl (e.g. vinyl esters), alkoxyalkyl (e.g. methoxymethyl esters), alkylthioalkyl (e.g. methylthiomethyl esters), alkanoyloxyalkyl (e.g. pivaloyloxymethyl esters), and substituted or unsubstituted aminomethyl esters (e.g. 2-dimethylaminoethyl esters). Additionally, any physiologically acceptable equivalents of the compounds of general structural formula I, similar to the metabolically labile esters, which are capable of producing the parent compounds of general Formula I in vivo, are within the scope of this invention.

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder.

The combination of compounds is preferably a synergistic combination. “Pharmaceutically acceptable salts” refer to derivatives of the disclosed compounds wherein the parent compound is modified by making an acid or base salts thereof. Examples of the pharmaceutically acceptable salts include, but not limited to, mineral or organic acid salts of the basic residues. The pharmaceutically acceptable salts include but not limited to N-methyl-D-glutamine, or 1-deoxy-1-methylamino-D-glucitol, or megluamine salt, HCl, HBr, HI, potassium (K), sodium (Na), calcium (Ca), magnesium (Mg), acetic, trifluoroacetic, citric, ascorbic, benzoin, methanesulfonic (mesylate), benzenesulfonic, bicarbonic, carbonic, ethane disulfonic, edetic, fumaric, maleic, lactic, malic, mandelic, gluconic, glutamic, glycolic, glycollyarsanilic, lauryl, hexylresorcinic, hyrdabamic, hydroxymaleic, hydroxynaphthoic, isethionic, lactobionic, napsylic, nitric, oxalic, pamoic, pantothenic, phenyllacetic, phosphoric, polygalacturonic, propionic, salicyclic, stearic, subacetic, succinic, sulfamic, sulfanilic, sulfuric, tannic, tartaric, tolouenesulfonic, and p-bromobenzenesulfonic.

### Definitions

For purposes of the present invention, as described and claimed herein, the following terms are defined as follows:

As used herein, the terms “comprising” and “including” are used in their open, non-limiting sense. The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups, which may be present in the compounds of formula I. The compounds of formula I that are basic in nature are capable of forming a wide variety of salts with various inorganic and organic acids. The acids that may be used to prepare pharmaceutically acceptable acid addition salts of such basic compounds of formula I are those that form non-toxic acid addition salts, i.e. salts containing pharmacologically acceptable anions, such as the N-methyl-D-glutamine, or 1-deoxy-1-methylamino-D-glucitol, or megluamine, acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

Certain compounds of formula I may have asymmetric centers and therefore exist in different enantiomeric forms. All optical isomers and stereoisomers of the compounds of formula I and mixtures thereof, are considered to be within the scope of the invention. With respect to the compounds of formula I the invention includes the use of a racemate, one or more enantiomeric forms, one or more diastereomeric forms, or mixtures thereof. The compounds of formula I may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

This invention also encompasses pharmaceutical compositions containing compounds of the formula I. and methods of treating ATTR, and ATTR-mediated diseases through administering prodrugs of compounds of the formula I. Compounds of formula I having carboxylic or amino groupscan be converted into prodrugs. Prodrugs include compounds wherein a carboxyl or an amino group can be derivatized into an easily cleavable group under physiological conditions in the mammals' bodies to enhance drug molecules absorption and increase its therapeutic effect in the subjects (e.g. human).

For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The compounds of the invention can also be used in combination with other drugs. If a compound used in the method of the invention is a base, a desired salt may be prepared by any suitable method known to the art, including treatment of the free base with an inorganic acid (such as hydrochloric acid, hydrobromic acid, sulfuric acid, nitric acid phosphoric acid, and the like), or with an organic acid (such as acetic acid, maleic acid, succinic acid, mandelic acid, fumaric acid, malonic acid, pyruvic acid, oxalic acid, glycolic acid, salicylic acid, pyranosidyl acid (such as glucuronic acid or galacturonic acid), alpha-hydroxy acid (such as citric acid or tartaric acid), amino acid (such as aspartic acid or glutamic acid), aromatic acid (such as benzoic acid or cinnamic acid), sulfonic acid (such as p-toluenesulfonic acid or ethanesulfonic acid), and the like. In the case of compounds of the present invention, prodrugs, salts, or solvates that are solids, it is understood by those skilled in the art that the hydroxamate compound, prodrugs, salts, and solvates used in the method of the invention, may exist in different polymorph or crystal forms, all of which are intended to be within the scope of the present invention and specified formulas. In addition, the hydroxamate compound, salts, prodrugs and solvates used in the method of the invention may exist as tautomers, all of which are intended to be within the broad scope of the present invention. Solubilizing agents may also be used with the compounds of the invention to increase the compounds solubility in water or physiologically acceptable solutions. These solubilizing agents include cyclodextrans, propylene glycol, diethylacetamide, polyethylene glycol, Tween, ethanol and micelle forming agents. Other solubilizing agents are cyclodextrans, particularly beta cyclodextrans and in particular hydroxypropyl betacyclodextran and sulfobutylether betacyclodextran. In some cases, the compounds, salts, prodrugs used in the method of the invention may exist as solvates. All such solvates, and combinations and mixtures thereof are intended to be within the broad scope of the present invention.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. In a preferred embodiment of the present invention, “treating” or “treatment” means at least the mitigation of a disease condition in a human that is alleviated by the inhibition of the activity related to transthyretin or other related protein, protein misfolding, protein fragments, accumulation, fibril formation, aggregation, and causing any dysfunction and/or damage in and/or to tissues and organs.

The present invention also includes prophylactic methods, comprising administering an effective amount of a compound of the invention, or a pharmaceutically acceptable salt, prodrug, pharmaceutically active metabolite, or solvate thereof to a mammal, such as a human, at risk for infection by a coronavirus. According to certain preferred embodiments, an effective amount of one or more compounds of the invention, or a pharmaceutically acceptable salt, prodrug, pharmaceutically active metabolite, or solvate thereof is administered to a human at risk for cardiovascular, kidney, and nervous system by ATTR. The prophylactic methods of the invention include the use of one or more of the compounds in the invention in any conventionally acceptable manner.

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder.

The combination of compounds is preferably a synergistic combination. “Pharmaceutically acceptable salts” refer to derivatives of the disclosed compounds wherein the parent compound is modified by making acid or base salts thereof. Examples of the pharmaceutically acceptable salts include, but not limited to, mineral or organic acid salts of the basic residues. The pharmaceutically acceptable salts include but not limited to HCl, HBr, HI, potassium (K), sodium (Na), calcium (Ca), magnesium (Mg), acetic, trifluoroacetic, citric, ascorbic, benzoin, methanesulfonic (mesylate), benzenesulfonic, bicarbonic, carbonic, ethane disulfonic, edetic, fumaric, maleic, lactic, malic, mandelic, gluconic, glutamic, glycolic, glycollyarsanilic, lauryl, hexylresorcinic, hyrdabamic, hydroxymaleic, hydroxynaphthoic, isethionic, lactobionic, napsylic, nitric, oxalic, pamoic, pantothenic, phenyllacetic, phosphoric, polygalacturonic, propionic, salicyclic, stearic, subacetic, succinic, sulfamic, sulfanilic, sulfuric, tannic, tartaric, tolouenesulfonic, and p-bromobenzenesulfonic.

### Synthesis of Compounds

Preparation of compounds of chemical structural formula I described below is general. It should be understood by those skilled in the art of chemical synthesis that some functional groups may not be compatible for certain synthetic routes and those cases may require appropriate changes including alternative starting materials, building blocks, intermediates, synthesis, synthetic methods process, appropriate sequence of synthetic steps, and compatible protection and deprotection strategy should be employed. The particular reaction conditions, such as reagents, solvents, temperature, and reaction time, should be used for conducting a synthetic reaction consistent with the nature of the functionality of the reactant and products involved. Those skilled in the art will recognize or be able to ascertain using no more than routine experimentation, numerous equivalents to the specific reagents can be utilized to produce compounds of the invention. Numerous modifications and variations of the present invention are possible and therefore it is understood that within the scope of the appended claims, the invention may be practiced otherwise that as specifically described herein. Other aspects, advantages and modifications are within the scope of the invention.

The synthesis of the novel deuterium-enriched (deuterated or deuterium-containing) compounds of chemical structural formula I of this invention is described below.

One of the two key building blocks for the synthesis of the target compounds of this invention is prepared from readily available 3,5-dichloroaniline 1 as illustrated below in Scheme 1. A mixture of 1, deuterium oxide (D₂O) and concentrated HCl (1 equivalent) is subjected to microwave irradiation or heating in a microwave at 180° C. for 30 min, yields 2,4,6-trideutero-3,5-dichloroaniline 2, after basic workup of the reaction. The deuterated aniline derivative 2 is converted into its corresponding nitrile derivative 3 via its diazonium salt, which is prepared by treatment of 2 with sodium nitrite (NaNO₂) in aqueous HCl at 0°−5° C. The low temperature 0°-5° C. is maintained by using ice-salt mixture as bath for the reaction flask. The diazonium salt obtained as such in situ is treated with cuprous cyanide (CuCN) at 0° C. in toluene for an hour, and then allowed to warm up to room temperature and stirred for 3 hours to produce the corresponding deuterated 3,5-dichlorobenzonitrile-d₃ 3 (Clarke et al. Org. Synth. 1925, 4, 69). Hydrolysis of 3 with HCl or H₂SO₄ in EtOH or MeOH produces the corresponding benzoic acid derivative, which upon its treatment with thionyl chloride (SOCl₂) in pyridine gives the desired intermediate building block, 2,4,6-trideutero-3,5-dichlorobenzoyl chloride 4 (Scheme 1).

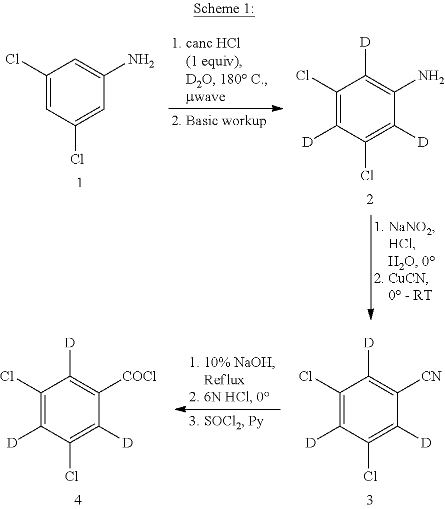 [SMILES: Nc1cc(Cl)cc(Cl)c1 ; [2H]c1c(N)c([2H])c(Cl)c([2H])c1Cl ; [2H]c1c(Cl)c([2H])c(C(=O)Cl)c([2H])c1Cl ; [2H]c1c(Cl)c([2H])c(C#N)c([2H])c1Cl]

General synthesis of compounds of chemical structural formula I from readily available 4-amino-3-hydroxybenzoic acid 5 (R═H) is described in Schemes 2 below. N-Acylation of 5 with the trideuterated-3,5-dichlorobenzoyl chloride 4 using triethylamine (Et₃N) or pyridine in THF or dichloromethane (CH₂Cl₂) produces the coupled amide product 6 as shown in Scheme 2. Heating a stirring mixture of 6 and p-toluene sulfonic acid (TsOH) in xylene or toluene overnight at reflux affords the cyclized 6-carboxybenzoxazole 7. Treatment of a solution of the benzoxazole carboxylic acid 7 in a mixture of toluene and methanol (3:2) with ethereal solution of trimethylsilyl diazomethane (Me₃SiCHN₂ or TMSCHN₂) produces the corresponding methyl ester 8. Methylation of carboxyl group of 7 to 8 is also realized by its treatment with diazomethane (CH₂N₂). Trimethlysilyl diazomethane (Me₃SiCHN₂) is preferred to diazomethane (CH₂N₂) as a convenient agent for conversion of COOH to COOCH₃ group. Saponification of the carbometoxy-benzoxazole 8 with aqueous sodium hydroxide (NaOH) or aqueous lithium hydroxide (LiOH) in THF-methanol affords the deuterium-enriched 6-carboxy-3,5-dichlorobenzoxazole-d₃, 9. Similarly, using various synthesized novel deuterium-enriched (deuterated) 4-amino-3-hydroxybenzoic acid analogs 5 and by following this general procedure described above and illustrated in Scheme 2, novel deuterium-enriched compounds of the present invention of chemical formula I are prepared. Treatment of the carboxylic acid 9 with N-methyl-D-glutamine (1-deoxy-1-methylamino-D-glucitol) yields the deuterium-enriched 6-carboxy-2-(3,5-dichlorophenyl)benzoxazole megluamine salt,10, a pharmaceutical salt of a compound of the present invention represented by the chemical structure I.

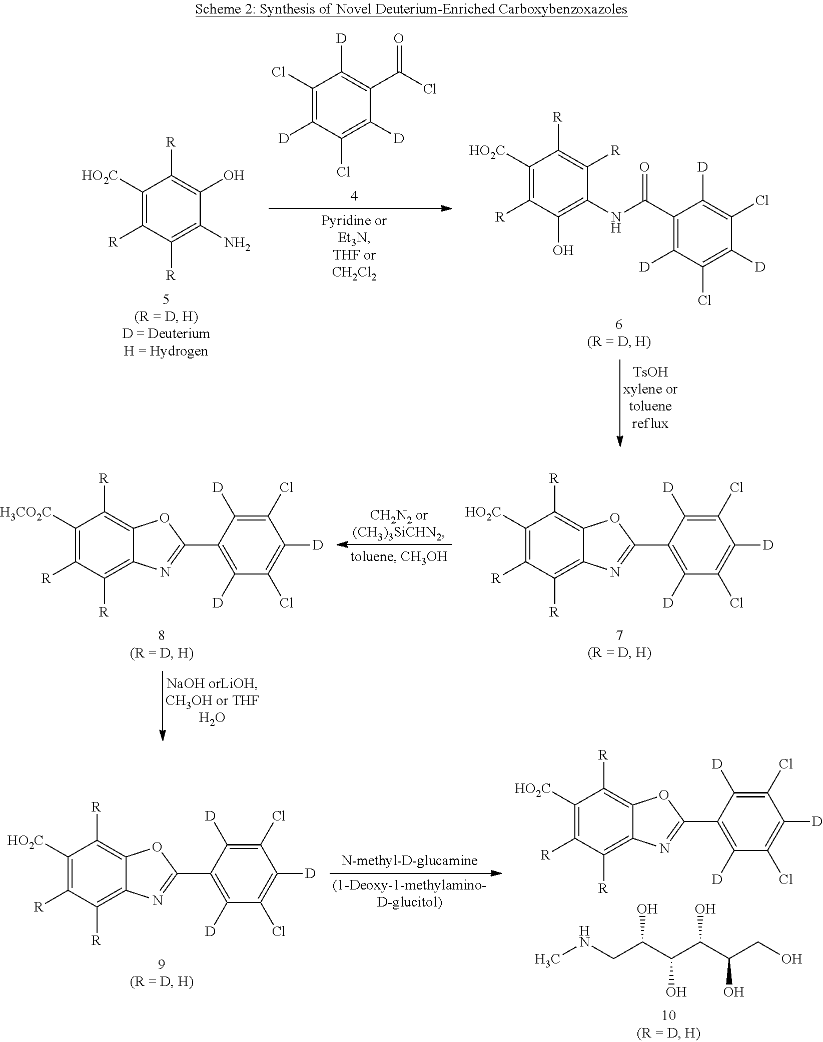

### Synthesis from Methyl 4-Amino-3-Hydroxybenzoate 11

Deuterium-enriched 6-carboxy-2-(3,5-dichlorophenyl)benzoxazole 9 and its megluamine salt 10, novel compounds of the present invention of chemical structural formula I are also synthesized from methyl 4-Amino-3-hydroxybenzoate 11 as shown below in Scheme 3. One of the advantages of this synthetic method starting from the methyl ester 11 is that it does not require the use of diazomethane (CH₂N₂) or Me₃SiCHN₂ (trimethylsilyl)diazomethane for conversion of the carboxylic acid derivative 7 to methyl ester 8 (Scheme 2). Heating at reflux of 4-amino-3-hydroxybenzoic acid 5 in methanol and a catalytic amount of sulfuric acid (CH₃OH/H₂SO₄) overnight yields Methyl 4-amino-3-hydroxybenzoate 11 (Scheme 3). Use of EtOH instead of MeOH provides the ethyl ester analog of 11. In addition, the use of HCl instead of H₂SO₄ as the acid catalyst produces the methyl or the ethyl esters, 11, when the mixture is refluxed in methanol or ethanol respectively. Similar to the general methodology described above and shown in Scheme 2, N-acylation of Methyl 4-amino-3-hydroxybenzoic acid 11 with the 2,4,6-trideuterated-3,5-dichlorobenzoyl chloride 4 using pyridine or triethylamine (Et₃N) in dichloromethane (CH₂Cl₂) or THF produces the coupled product 12. Heating of 12 with p-toluene sulfonic acid monohydrate (TsOH) or methanesulfonic acid (CH₃SO₂H), in xylene or toluene at reflux overnight produces its cyclized product deuterium-enriched 6-carbomethoxy benzoxazole 8. This conversion of 12 to 8 is also achieved by microwave irradiation at high temperature for ½-1 h in a microwave apparatus in lieu of overnight reflux. Saponification of novel deuterated compounds 8 by treatment with aqueous solution of NaOH or LiOH in methanol and tetrahydrofuran (THF) gives the novel deuterium-enriched product 6-carboxy-2-(3,5-dichlorophenyl-2,4,6-d₃)benzoxazole 9. Treatment of the deuterium-enriched 6-carboxy-3,5-dichlobenzoxazole-d₃ 9 with N-methyl-D-glutamine (1-deoxy-1-methylamino-D-glucitol) yields 10, the deuterium-enriched 6-carboxy-2-(3,5-dichlorophenyl-2,4,6-d₃)benzoxazole megluamine salt of 9 (Scheme 3), a pharmaceutical salt of a compound of the present invention represented by the chemical structure I.

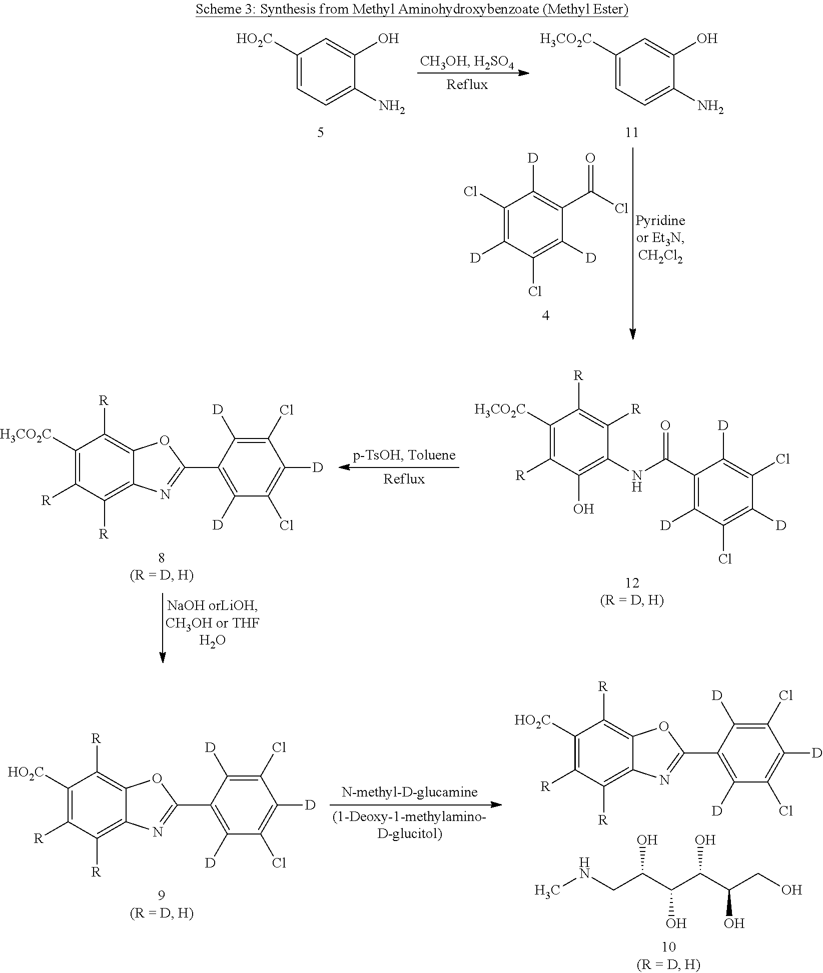

Novel deuterium incorporation in benzoxazoles 8 and 9 is further achieved by microwave synthesis as shown in Scheme 4. Microwave irradiation of a mixture of 8 and deuterium oxide (D₂O) and 1 equivalent of concentrated HCl at 180° C. for 30 min in a microwave apparatus produces further deuterated benzoxazole 8-d₄. Similarly, microwave heating of a mixture of carboxybenzoxazole 9 with D₂O and 1 equivalent of concentrated HCl at 180° C. for 30 min gives the deuterated compound 944 as shown in scheme 4.

Microwave irradiation (microwave heating) of a mixture of 6-d₃ with deuterium oxide (D₂O) and deuterium chloride (DCl) at pD 0.32 (pD is equivalent of pH in case of HCl) for 30 min yields the corresponding deuterated novel compound, 4-amido-3-hydroxybenzoic acid derivative 6-dn. Similarly, microwave irradiation of a mixture of methyl ester 7 (7-d₃) in D₂O and DCl at pD of 0.32 affords novel deuterium-enriched methyl amidohydroxybenzoate 7-dn as shown below in Scheme 4.

Heating under reflux conditions of a mixture of carboxyphenol compound 6, with deuterium oxide (D₂O) and deuterium chloride (DCl) at pD 0.32 under N₂ atmosphere for 1 h yields the corresponding deuterated carboxyphenol 6-dn. Similarly, refluxing a mixture of 4-amino-3-hydroxybenzoic acid 5 in D₂O and DCl at pD of 0.32 under N₂ for 1 h gives the corresponding deuterium-enriched product 5-dn as shown in Scheme 5. Microwave irradiation of 5 and 11 gives deuterated 5-d and 11-d respectively also as shown in Scheme 5.

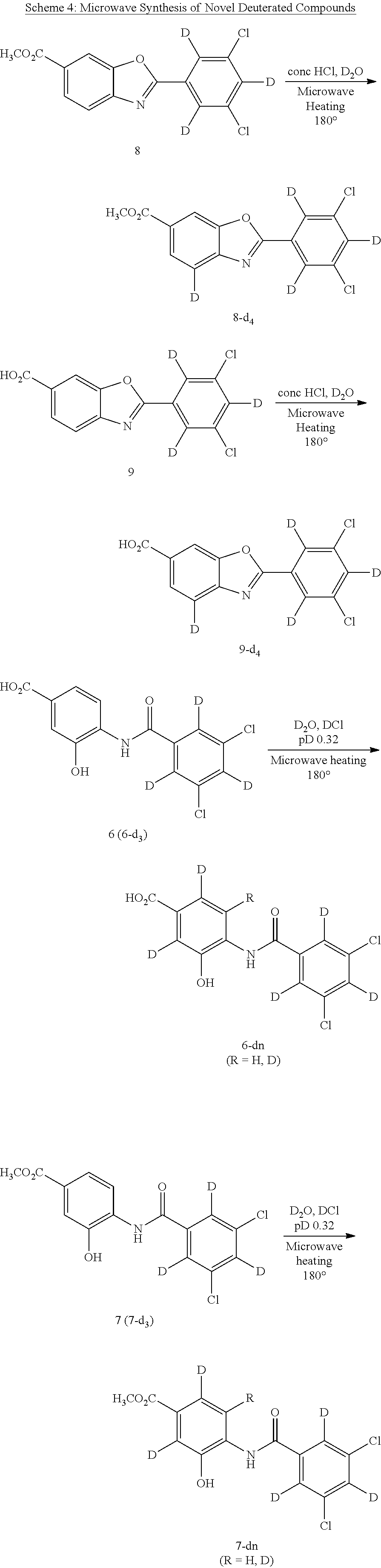

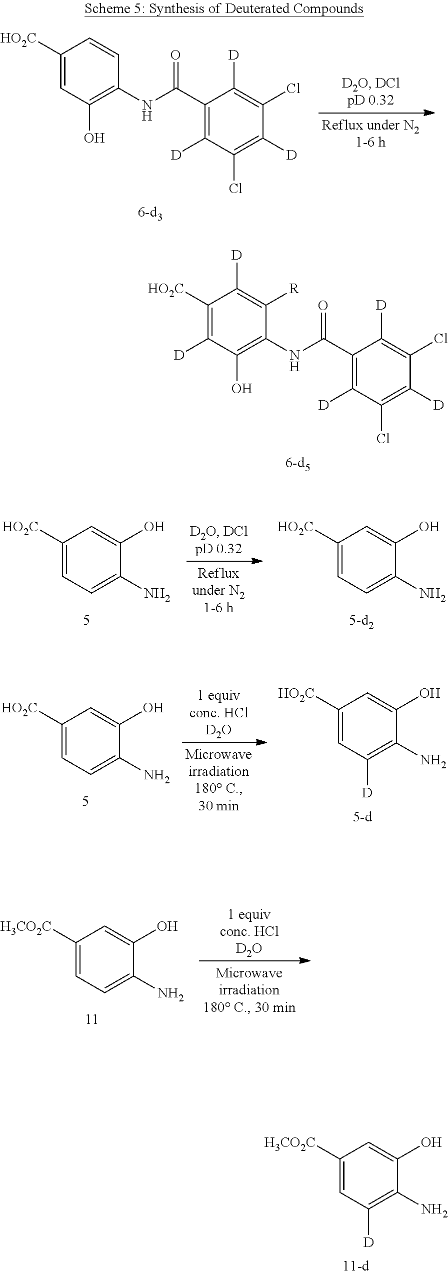

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, s or sec, second(s); min, minute(s); h or hr, hour(s); Room Temperature. RT, rt, etc.

### Materials, Reagents, Methods and Instruments:

Prealbumin from human plasma (human TTR) is purchased from Sigma. Diflunisal, Thyroxine (T4), and resveratrol are purchased from Fisher. All reactions are carried out under argon atmosphere using dry solvents under anhydrous conditions, unless otherwise noted. The solvents used are ACS grade from Fisher. Reagents are purchased from Aldrich and Acros, and used without further purification. Reactions are monitored by thin-layer chromatography (TLC) carried out on 0.20 mm POLYGRAM® SIL silica gel plates (Art.-Nr. 805 023) with fluorescent indicator UV254 using UV light as a visualizing agent. Normal phase flash column chromatography is carried out using Davisil® silica gel (100-200 mesh, Fisher). Wild type TFR concentration is serum, is measured at Stanford Medical School using nephelometric analyzer (28 mg/dL or 5 μM).

Unless stated otherwise, all reactions are conducted in oven-dried glassware under a dry nitrogen (N₂) or argon (Ar) atmosphere. All solvents and reagents are purchased from Aldrich (sigma-Aldrich) or Fisher and are used as obtained without further purification. Thin Layer Chromatography (TLC) analysis are performed using commercially available TLC plates.

### Examples

### Preparation of Deuterated-3,5-dichloroaniline-d₃ 2

Step A: A mixture of 3,5-dichloroaniline 1 (1.0 g, 6.17 mmol) and 1 equivalent of conc HCl in D₂O is micro irradiated (□ wave) at 180° C. in microwave apparatus for 30 minutes (Martins, A., Lautens, M., Org. Lett. 2008, 10, 4351-4353). The reaction mixture is allowed to attain room temperature and quenched cautiously with slow addition of saturated aqueous solution of NaOH. When the mixture is basic, the deuterated hydroxyaniline is extracted three times (3×) with CH₂Cl₂. The combined organic solvent are dried over anhydrous Na₂SO₄, filtered and concentrated in vacuo to yield trideuterated 2 (0.83 g, 81%).

### Deuterated 3,5-Dichlorobenzonitrile-d₃ 3

To a stirred solution of 2 (0.83 g) in HCl at 0° C. is added NaNO₂ and the mixture stirred for an hour to produce a corresponding diazonium salt in situ. Cuprous cyanide (CuCN, 1.5 equiv) is added to the reaction mixture and stirred for 2 hours at 0° C. and for 3 hours at room temperature. The mixture is diluted with methylene chloride and ether and then filtered through celite. The organic phase is separated and dried over anhydrous Na₂SO₄, filtered and concentrated to yield deuterated 3 (0.75 g, 85%). Use of KCN instead of CuCN is also used to convert the diazonium salt to the 3,5dichlorobenzonitrile-d₃ 3.

### Deuterated 3,5-Dichlorobenzoyl Chloride-d₄ 4

Deuterated benzonitrile 3 (0.75 g, 4.285 mmol) is heated under reflux conditions with enough aqueous 10% NaOH for several hours until hydrolysis is complete. The mixture is then cooled to 0° C. and acidified with HCl, extracted with CH₂Cl₂ (3×), combined extracts filtered, dried over Na₂SO₄ and concentrated in vacuo to yield 3,5-dichlorobenzoic acid-d₃, which is subjected to its thionylation (SOCl₂/Py) in the next step.

To a stirred solution of 3,5-dichlorobenzoic acid-d₃ in CH₂Cl₂ or THF is added pyridine (3.5 equiv) followed by addition of SOCl₂ (1 equiv) and mixture stirred at room temperature for 8 h, then refluxed for 1 h, and concentrated in vacuo to afford the desired 2,4,6-trideutero-3,5-dichlorobenzoyl chloride 4 (0.74 g, 90%) as shown in Scheme 1, and used in the next reaction step without any further purification.

### Deuterated 4-(3,5-dichlorobenzamido-2,4,6-d₃)-3-hydroxybenzoic acid 6

Step B: To a stirred mixture of 4-amino-3-hydroxybenzoic acid 5 (0.53 g, 3.48 mmol) and pyridine (5 equiv) (or Et₃N) in 10 mL of THF or dichloromethane (CH₂Cl₂) is added 2,4,6-trideuterated-3,5-dichlorobenzoyl chloride 4 (0.74 g, 3.48 mmol) at room temperature and the reaction mixture stirred overnight (18 h), refluxed for 1 h. After completion of N-acylation of 5, the mixture is acidified with 1N HCl and extracted 3× times with (CH₂Cl₂), 3× with ethyl acetate (EtOAc)/ether (Et₂O). The combined organic extracts are washed 1× with brine, dried over anhydrous Na₂SO₄ or MgSO₄, filtered and concentrated in vacuo to yield couple product 6 (1.1 g, 96%) as shown in Scheme 2. Mass Spec MS: m/e: 329 (M+1).

### Deuterated 6-Carboxy-2-(3,5-dichlorophenyl-2,4,6-d₃)benzoxazole 7

Step C: A stirred mixture of 6 (150 mg, 0.455 mmol) and p-toluene sulfonic acid (pTsOH, 1.5 equiv) or methane sulfonic acid (1.5 equiv) in xylene or toluene is refluxed overnight (>14 h). The reaction mixture is allowed to cool to room temperature and aqueous 1N NaOH solution is added, the organic and aqueous layers are separated. The aqueous layer is then acidified with 1N HCl to pH 2, and then extracted sequentially with ethyl acetate (3×EtOAc) and 3×CH₂Cl₂. The combined organic phases are dried over anhydrous MgSO₄, filtered and concentrated in vacuo using rotary evaporator to give the cyclized 6-carboxybenzoxazole-d₃ 7 (140 mg, 98%). Mass Spec MS: calculated for C₁₄H₄D₃Cl₂NO₃ (M+1), 310.9991, found 310.9990.

Rapid conversion of acyclic intermediate 6 to cyclic benzoxaole 7 is also accomplished using microwave synthesis. Microwave irradiation (heating in a microwave apparatus) of a mixture of 6 and TsOH in toluene at 180° C. for 30 minutes, after reaction work-up as described above affords the deutearted benzoxazole 7.

### Deuterated 6-Carbomethoxy-2-(3,5-dichlorophenyl-2,4,6-d₃)benzoxazole 8

To a solution of the 6-carboxybenzoxazole-d₃ 7 (140 mg, 0.45 mmol) in toluene and methanol (3:2), is added 2 equivalents of hexane solution of trimethylsilyldiazomethane (Me₃SiCHN₂ or TMSCHN₂, 2 equiv) and mixture is stirred for 1 h to produce the corresponding methyl ester benzoxazole 8, which is purified by flash column chromatography over silica gel and eluted with hexanes, and then followed by 5, 10, 20, 25, 30% ethyl acetate in hexanes to yield 8 (135 mg, 92%). Mass Spec MS: calculated for C₁₅H₆D₃Cl₂NO₃ (M+1), 325.0148, found 325.0147.

Using diazomethane (generated form commercially available precursor N-methyl-N-tolylsulfonyl)nitrosamide or Diazald) instead of TMSCHN₂ as a methylating agent also gives the desired 6-carbomethoxy-2-(3,5-dichlorophenyl-2,4,6-d₃)benzoxazole 8 (138 mg, 95%) as shown in Scheme 2. Mass Spec MS: calculated for C₁₅H₆D₃Cl₂NO₃ (M+1), 325.0148, found 325.0146.

### Deuterated 6-Carboxy-2-(3,5-dichlorophenyl-2,4,6-d₃)benzoxazole 9

Step D: To a solution of deuterium-enriched carbomethoxybenzoxazole 8 (135 mg, 0.415 mmol) in 3:1:1 solvent mixture of THF/CH₃OH/H₂O (5 mL) is added 5 equivalents of an aqueous solution of sodium hydroxide (NaOH in H₂O) or an aqueous solution of lithium hydroxide (LiOH in H₂O) and resulting mixture stirred until completion of ester hydrolysis. After completion of saponification (monitored by TLC), 1N HCl is added to the acidify reaction mixture to pH 2. The product is extracted with ethyl acetate (3×) and CH₂Cl₂ (3×), the combined organic extracts dried over anhydrous MgSO₄, filtered and concentrated in vacuo to yield deuterium-enriched 6-carboxy-2-(3,5-dichlorophenyl-2,4,6-d₃)benzoxazole 9 (0.12 g, 94%). Mass Spec MS: calculated for C₁₄H₄D₃Cl₂NO₃ (M+1), 310.9991, found 310.9990.

### Deuterium-enriched 6-Carboxy-2-(3,5-dichlorophenyl-2,4,6-d₃)benzoxazole megluamine,10

Step E: To a solution of 6-Carboxy-2-(3,5-dichlorophenyl-2,4,6-d₃)benzoxazole 9 (100 mg, 0.32 mmol) in isopropanol, (ethanol, methanol or CH₂Cl₂) is added N-methyl-D-glutamine (1-deoxy-1-methylamino-D-glucitol or megluamine, 63 mg, 1 equiv) and the resulting mixture stirred for 1-2 h to ensure salt formation and then concentrated in vacuo using rotary evaporator to give 10, deuterium-enriched 6-carboxy-2-(3,5-dichlorophenyl)benzoxazole megluamine salt of 9, compounds of the present invention represented by the chemical structural I.

### Methyl 4-Amino-3-hydroxybenzoate 11

A catalytic amount of concentrated H₂SO₄ is added to a stirred solution of 4-Amino-3-hydroxybenzoic acid 5 (5 g, 32.7 mmol) in anhydrous methanol and the reaction mixture refluxed overnight. Methanol is removed in vacuo and the residue dissolved in a mixture of methylene chloride, ether and ethyl acetate. The organic phase is washed with saturated aqueous solution of sodium bicarbonate (NaHCO₃), then brine, and dried over anhydrous MgSO₄, filtered and concentrated in vacuo using rotary evaporator to yield the methyl ester ester 11 (5.1 g, 93%). Mass Spec: m/z 168 (M+1).

Use of ethanol (EtOH) instead of methanol (MeOH) provides the ethyl ester. Also, the use of HCl instead of H₂SO₄ produces the methyl or the ethyl ester when the mixture is refluxed in methanol or ethanol respectively.

### Methyl 4-(3,5-dichlorobenzamido-2,4,6-d₃)-3-hydroxybenzoate 12

Using the same procedure as described in Step B above, the deuterated intermediate Methyl 4-(3,5-dichlorobenzamido-2,4,6-d₃)-3-hydroxybenzoate 12 is prepared from 11 and trideuterated 3,5-dichlorobenozyl-(2,4,6-d₃) chloride 4 as shown in Scheme 3. N-bezoylation of 11(100 mg, 0.6 mmol) with 4 (127 mg, 1 equiv) by following Step B method affords 12 (195 mg, 95%).

### Deuterated 6-Carbomethoxy-2-(3,5-dichlorophenyl-2,4,6-d₃)benzoxazole 8

Using the synthetic method as described above in Step C, refluxing 12 (150 mg, 0.437 mmol) with TsOH or methane sulfonic acid (1.5 equiv) in toluene or xylene overnight gives deuterated 6-Carbomethoxy-2-(3,5-dichlorophenyl-2,4,6-d₃)benzoxazole 8 (135 mg, 95%) as shown in Scheme 3. Mass Spec MS: calculated for C₁₅H₆D₃Cl₂NO₃ (M+1), 325.0148, found 325.0149.

### Deuterated 6-Carboxy-2-(3,5-dichlorophenyl-2,4,6-d₃)benzoxazole 9

Using the synthetic procedure as described above in Step D, 6-carbomethoxybenzoate 8 (130 mg, 0.4 mmol) is converted to its corresponding acid 6-carbomethoxy-2-(3,5-dichlorophenyl-2,4,6-d₃)benzoxazole 9 (119 mg, 96%) as shown in Scheme 3. Mass Spec MS: calculated for C₁₅H₆D₃Cl₂NO₃ (M+1), 325.0148, found 325.0147.

### Deuterium-enriched 6-Carboxy-2-(3,5-dichlorophenyl-2,4,6-d₃)benzoxazole megluamine,10

To a solution of 6-Carboxy-2-(3,5-dichlorophenyl-2,4,6-d₃)benzoxazole 9 (30 mg, 0.0964 mmol) in isopropanol, (ethanol, methanol or CH₂Cl₂) is added N-methyl-D-glutamine (1-deoxy-1-methylamino-D-glucitol or megluamine, 19 mg, 1 equiv) and the resulting mixture stirred for 1-2 h to ensure salt formation and then concentrated in vacuo using rotary evaporator to give the deuterium-enriched 6-carboxy-2-(3,5-dichlorophenyl)benzoxazole megluamine salt 10 as shown in Scheme 3.

### Deuterated 6-Carbomethoxy-2-(3,5-dichlorophenyl-2,4,6-d₃)benzoxazole-4-d₁, 8-d₄

Using the microwave synthesis procedure as described in method Step A above, tetradeuterated 6-Carbomethoxy-2-(3,5-dichlorophenyl-2,4,6-d₃)benzoxazole-4-d₁, 8 (12 mg) is prepared from the corresponding trideuterated 6-carboxybenzoxazole-d₃, 8 as shown in Scheme 4.

### Deuterated 6-Carboxy-2-(3,5-dichlorophenyl-2,4,6-d₃)benzoxazole-4-d₁ 9-d₄

Deuterium-enriched tetradeutertaed 6-carboxy-2-(3,5-dichlorophenyl-2,4,6-d₃)benzoxazole-4-d₁ 9-d₄ is prepared from 9 using microwave synthesis according to general procedure described in Step A and shown in Scheme 4 above to give 9-d₄ (14.5 mg).

### 4-Amino-3-hydroxybenzoic-5-d₁ acid 5-d

Microwave irradiation of 4-Amino-3-hydroxybenzoic acid 5-d in D₂O and conc. HCl at 180° C. for 30 min according to the general procedure described in Step A and as shown in Scheme 5 above yields the deuterated 4-Amino-3-hydroxybenzoic acid 5-d (55 mg).

### Methyl 4-Amino-3-hydroxybenzoate-5-d₁ 11-d

Microwave irradiation of methyl 4-Amino-3-hydroxybenzoate 11 in D₂O and conc HCl at 180° C. for 30 min according to the general procedure described in Step A and as shown in Scheme 5 above produces the deuterated methyl 4-Amino-3-hydroxybenzoate-5-d₁ 11 (48 mg).

### Deuterated 4-(3,5-dichlorobenzamido-2,4,6-d₃)-3-hydroxybenzoic acid-2,6-d₂, 6-dn

Step F: Deuterium oxide (D₂O, 10 g) is added to 6-d₃ (100 mg, 0.3 mmol) and the mixture stirred. Then deuterium chloride (DCl) is added to the mixture to adjust pD (pH) of the reaction mixture to 0.32 and the resulting mixture is heated at 180° C. for 30 min by microwave irradiation in a microwave synthesis apparatus as in Step A and shown in Scheme 4 above. The resulting mixture is lyophilized to remove D₂O and DCl to yield deuterated 4-(3,5-dichlorobenzamido-2,4,6-d₃)-3-hydroxybenzoic acid-2,6-d₂, 6-dn (75 mg, 75%).

### Methyl 4-(3,5-dichlorobenzamido-2,4,6-d₃)-3-hydroxybenzoate-2,6-d₂, 7-dn

Deuterated Methyl 4-(3,5-dichlorobenzamido-2,4,6-d₃)-3-hydroxybenzoate 2,6-d₂, 7-dn is prepared from its precursor 7-d₃ (100 mg, 0.29 mmol) according to the procedure described in Step F and as shown in Scheme 4 above to give the product 7-dn (78 mg, 78%).

### Deuterated 4-(3,5-dichlorobenzamido-2,4,6-d₃)-3-hydroxybenzoic acid-2,6-d₂, 6-d₅

Step G: Deuterium oxide (D₂O, 100 g, 5 moles) is added to 4-Fluoro-3-hydroxybenzoic acid (lg, 0.0064 mole). Then deuterium chloride (DCl) is added to the mixture to adjust the pD (pH) of the reaction mixture to 0.32 and the resulting mixture is refluxed under nitrogen (N₂) for 1-2 h. The resulting mixture is lyophilized to remove D₂O and DCl to yield 6-d₅ (70 mg, 70%) as shown in Scheme 5.

### Deuterated 4-Amino-3-hydroxybenzoic-2,6-d₂ acid, 5-d₂

Deuterated 4-Amino-3-hydroxybenzoic-2,6-d₂ acid, 5-d₂, is synthesized from commercially available 4-amino-3-hydroxybenzoic acid 5 (153 mg, 1 mmol) according to the procedure described in Step G and shown in Scheme 5 above to give 5-d₂ (0.11 mg, 71%).

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth in this invention.

### Biological Assays

Biological assays and methods for evaluating the activity of compounds of the present invention of general chemical structural formula I, and their pharmaceuticals salts, to increase the stability of tetrameric protein transthyretin (TTR) thereby preventing it from misfolding, aggregating, and depositing into organs and tissues and forming TTR amyloid are described below.

### Binding Affinity Assay of Compounds to TTR

The binding affinity of compounds of the present invention to Transthyretin (TTR) at physiological pH is determined using fluorescence polarization (FP) assay. Known TTR kinetic stabilizer compounds (TTR natural ligand T₄, tafamidis and acoramidis) are used as controls for comparison in this assay. The FP assay is a competitive assay that is used to determine ligand binding to TTR by determining displacement of a fluorescent probe from T₄-binding sites of TTR by test compounds. All test compounds bind to FUR (purified from human plasma) at 10 μM. Test compounds, are assayed in a multi-point dose-response FP assay (concentration range between 0.003 and 100 μM). Isothermal titration calorimetry (ITC) is used to determine the binding constants of test compounds to TTR and also to determine cooperativity between the two TTR T₄ sites. ITC measurements show high binding constant of test compounds in low nanomolar range. Analysis of the free energies associated with compounds shows high binding affinity and the dissociation constants show that compounds bind to TTR with negative cooperativity.

### Transthyretin Amyloid Fibril Formation Assay

The test compounds are dissolved in DMSO at a concentration of 720 μM. A solution of 5 microliter of a test compound is added to 0.5 mL of a 7.2 μM TTR solution in 10 mM phosphate pH 7.6, 100 mM KCl, 1 mM EDTA buffer, allowing the compound to incubate with TTR for 30 min. 495 μL of 0.2 mM acetate pH 4.2, 100 mM KCl, 1 mM EDTA is added, to afford final protein and inhibitor concentrations of 3.6 μM each and a pH of 4.4 The mixture is then incubated at 37° C. for 72 h, after which the tubes are vortexed for 3 seconds and the optical density is measured at 400 nm. The extent of fibril formation is determined by normalizing each optical density by that of TTR without inhibitor, defined to be 100% fibril formation. Control solutions of each compound in the absence of TTR are also tested and none absorbed appreciably at 400 nm.

### Isothermal Titration Calorimetry Assay

A 25 μM solution of a test compound (in 10 mM phosphate pH 7.6, 100 mM KCl, 1 mM EDTA, 8% DMSO,) is titrated into a 1.2 μM solution of TTR in an identical buffer using a Mierocal MCS Isothermal Titration Calorimeter (Microcal. Northampton, Mass.). An initial injection of 2 μL is followed by 25 injections of 10 μL at 25° C. The thermogram is integrated and a blank is subtracted to yield a binding isotherm that fit best to a model of two identical binding sites using the ITC data analysis package in ORIGIN 5.0 (Microcal).

### Isothermal Titration Calorimetry Assay

Calorimetric titrations are conducted on a VP-ITC calorimeter (MicroCal, Northhampton, Mass.). A solution of test compound (a compound of the present invention, and Tafamidis) (25 μM in PBS pH 7.4, 100 mM KCl, 1 mM EDTA, 8% DMSO) is prepared and degassed for 0.25 hour, and then titrated into an isothermal titration calorimetry cell containing 2 μM of TTR in an identical buffer. Solutions of test compounds (8.0 μL each) are injected into the isothermal titration calorimetry cell at 25° C. to fully saturate TTR with ligand. Integration of the thermogram after the subtraction of blanks gives a binding isotherm that fit best to a model of two interacting sites exhibiting negative cooperativity. The data are fit by a nonlinear least squares approach with four adjustable parameters: K_{d1}, ΔH1, K_{d2}, and ΔH2 using the isothermal titration calorimetry data analysis module in MicroCal ORIGIN 5.0 software.

### Isothermal Titration Calorimetry Assay

The thermodynamic parameters that characterize the binding of TTR ligands to WT-TTR are also determined using a MicroCal Auto-iTC200 Calorimeter (MicroCal, Malvern-Panalytical), [Francisca, P., et al. FEBS Journal, 2021, 288, 310-324]. A 100 μM solution of a test compound (in a PBS buffer pH 7.0 containing 100 mM KCl, 1 mM EDTA and 2.5% DMSO) is titrated into an ITC cell containing a 5 μM solution of WT-TTR in the same buffer at 25° C. A stirring speed of 750 rpm and 2 μL injections is programmed, with a 150 s equilibration period between each injection to allow the calorimetric signal (thermal power) to return to baseline and a 10 μcal/s reference power. Two independent titrations are done for each TTR ligand. The experimental data are analyzed with a general model for a protein with two ligand-binding sites [A unified framework based on the binding polynomial for characterizing biological systems by isothermal titration calorimetry, Vega, S., et al. Methods (Amsterdam, Netherlands), 2015, 76, 99-115; Isothermal titration calorimetry: general formalism using binding polynomials, Freire, E., et al., Methods in Enzymology, 2009, 455 (Biothermodynamics, Part A), 127-155] implemented in Origin 7.0 (OriginLab) accounting for cooperative and non-cooperative binding.

### Urea-Induced TTR Tetramer Dissociation Kinetics

TTR solutions (1.8 μM in PBS) are incubated with different TTR ligands (3.6 μM) for 30 min at RT, and 6 M urea is added. DMSO is used as a control. The process of unfolding is tracked by intrinsic fluorescence spectroscopy using FP-8200 Spectrofluorometer (Jasco). Trp residues are excited at 295 nm, and emission spectra are collected from 310 to 400 nm. Trp exposure upon denaturation red shifts the wavelength of maximum fluorescence from approximately 335 to 355 nm. The 355/335 fluorescence emission intensity is normalized from minimum (100% folded) to maximum (0% folded) and plotted as a function of time. The TTR fluorescence of the control sample after incubation at RT for 96 h in 6 M urea is considered the maximum.

### TTR In Vitro Aggregation Assay

The anti-aggregational activity of TTR ligands is evaluated using the established acid-mediated aggregation assay [Francisca, P., et al. FEBS Journal, 2021, 288, 310-324]. WT-TTR solutions (7 μM in 10 mM sodium phosphate, 100 mM KCl, 1 mM EDTA, 1 mM DTT, pH 7.0) are mixed with increasing concentrations of test compounds (prepared in 100% DMSO). The percentage of DMSO is adjusted to 5% (v/v) in the final reaction assay mixture. After incubating for 30 min at 37° C., the pH of the samples is dropped to 4.2 by the addition of acetate buffer (100 mM sodium acetate, 100 mM KCl, 1 mM EDTA, 1 mM DTT, pH 4.2), and the solutions are further incubated for 72 h at 37° C. The extent of TTR aggregation is determined by measuring turbidity at 340 nm using a Varian Cary Eclipse Fluorescence Spectrophotometer (Agilent Technologies). As some of the compounds present dose-dependent absorbance at 340 nm, each measurement is corrected with a buffer containing the same concentration of the test compound but lacking TTR. For each inhibitor concentration, the percentage of TTR aggregation is given by the ratio of the turbidity of the sample of interest to that of a control sample incubated without compound multiplied by 100%.

### TTR Stabilization Studies by Isoelectric Focusing

Isoelectric focusing (IEF) under semi-denaturing conditions [Binding of epigallocatechin-3-gallate to transthyretin modulates its amyloidogenicity, Ferreira, N., FEBS Letters, 2009, 583, 3569-3576] is used to evaluate the stabilizing effect of test compound of the present invention on recombinant TTR and in plasma TTR. Recombinant WT-TTR is produced using an Escherichia coli bacterial expression system, as detailed elsewhere [Selective binding to transthyretin and tetramer stabilization in serum from patients with familial amyloidotic polyneuropathy by an iodinated diflunisal derivative, Almeida, M. R., et al. Biochemical Journal, 2004, 381, 351-356]. For the plasma assays, human plasma from control individuals (n=6), carrying WT-TTR (≈3.9 μM), is incubated ON at 4° C. with tafamidis, tolcapone, or test compound of the present invention at two different concentrations (19.5 and 39 μM). Similarly, recombinant WT-TTR (6 μM) is also treated ON at 4° C. with the same compounds at concentrations of 30 and 60 μM. DMSO (5%) is used as vehicle. Control samples are incubated in similar conditions without the compounds. After incubation, the samples are loaded into a native PAGE, and the gel band containing TTR is excised and applied to an IEF gel. The IEF gel contained 4 M urea (semi-denaturing conditions) and 5% (v/v) ampholytes, pH 4-6.5 (Sigma-Merck), and is run at 1200 V for 5 h. Proteins are fixed and stained with Coomassie blue. The gels are scanned using a GS-900 calibrated densitometer (Bio-Rad) and analyzed by densitometry using the QuantityOne software version 4.6.6 (Bio-Rad). The ratio of the TTR tetramer over total TTR (TTR tetramer+monomer) is calculated for each plasma sample, and the percentage of tetramer stabilization is calculated as ((ratio treated sample−ratio control sample)/ratio control sample)×100. Treated and control plasma samples come from the same donor.

### T₄ Binding Competition Assay

Binding competition assays with radioactive T₄ (2-amino-3-(4-(4-hydroxy-3,5-diiodophenoxy)-3,5-diiodophenyl)propanoic acid) are performed by incubation of 5 μL of human plasma samples of WT-TTR carriers (n=4) with 1 μL of [¹²⁵I]-T₄ (specific radioactivity 1250 μCi/μg; concentration 320 μCi/mL; Perkin Elmer) in the presence of 39 μM compounds. The negative control is performed by addition of same amount of DMSO to samples. After 1 h incubation at RT, plasma proteins are fractionated by native PAGE [Transthyretin (prealbumin) in familial amyloidotic polyneuropathy: genetic and functional aspects, Saraiva, M. J., et al., Advances in neurology, 1988, 48, 189-200], and the gels are dried and exposed to an autoradiography film. The films are scanned, and the intensity of the bands is determined by densitometry using Image Lab software version 5.2.1 (Bio-Rad). The ratio of T₄ (2-amino-3-(4-(4-hydroxy-3,5-diiodophenoxy)-3,5-diiodophenyl)propanoic acid) bound to TTR over total T₄ (TBG+ALB+TTR) is calculated for each sample and normalized to the maximum value, which corresponds to the negative control sample.

### Biological Activity of Test Compounds as Stabilizers of Both WT-TTR and V122I-TTR in Human Serum Against Acid-Mediated Dissociation and Amyloidogenesis

The test compounds are tested for their ability to stabilize WT-TTR in human serum. TTR tetramer dissociation to monomers and subsequent aggregation occur very inefficiently at neutral pH. To measure the TTR stabilizing effect of test compounds, an acid-mediated tetramer dissociation assay is used. Test compounds (10 μM) are pre-incubated with human serum (TTR 5 μM) and the pH is lowered to pH 4.0 to induce aggregation. Aliquots, taken at 0 and 72 hours, are treated with glutaraldehyde to cross-link TTR tetramers that remained intact in the serum sample. SDS-PAGE followed by immunoblot analysis is used to measure the amount of intact TTR tetramer after 72 hours of acid treatment in the presence and absence of test compounds. Test compounds of the present invention are found to be highly effective stabilizers of serum WT-TTR. At 10 M, test compounds of the present invention are also highly effective in nearly complete stabilization of the V122I-TTR mutant in serum samples.

### Determination of Solubility of Compounds

Solubility of compounds is determined by addition of known and weighed amount of a compound of the present invention to a stirred distilled or deionized water until total complete dissolution. All experiments are performed in triplicate.

### Cytotoxicity Assay

Cytotoxicity assays are performed to evaluate chemical toxicity of test compounds of the present invention to human cells. Two cell lines, HeLa and HepG2 cells, are cultured in MEM ALPHA (Gibco) and 10% FBS at 37° C. in 5% CO₂ humidified atmosphere. HeLa cells are seeded at 3500 cells/well and HepG2 cells at 4500 cells/well in 96-well plates and incubated with increasing concentrations of compound at a range of concentrations from 2 to 100 μM during 72 h at 37° C. Controls are performed with the equivalent amount of DMSO relative to each concentration of compound diluted in MilliQ water. Then, cell viability is determined by addition of 10 μL of the PrestoBlue reagent (Thermo Fisher Scientific), and after an incubation period of 15 min at 37° C., the fluorescence intensity is collected using a 590/20 filter with an excitation wavelength of 535 in a Victor3 Multilabel Reader (PerkinElmer). Experiments are carried out in triplicate, and the percentage of cell viability for each well is calculated as (intensity sample−mean intensity blank)/(mean intensity control−mean intensity blank)×100, where “mean intensity blank” corresponds to the mean intensity of wells with PrestoBlue alone and “mean intensity control” is the mean intensity of wells that contain the corresponding percentage of DMSO.

What is claimed is:

1. A deuterium-enriched compound selected from the group consisting of:
(a) 2-(3,5-dichlorophenyl-2,4,6-d₃)-1,3-benzoxazole-6-carboxylic acid-d, 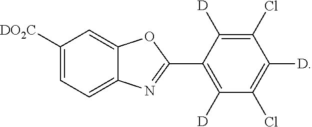
(b) 2-(3,5-dichlorophenyl-2,4,6-d₃)-1,3-benzoxazole-6-carboxylic acid, 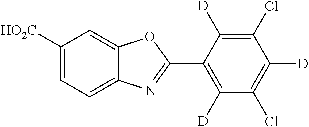 [SMILES: [2H]c1c(Cl)c([2H])c(-c2nc3ccc(C(=O)O)cc3o2)c([2H])c1Cl]
(c) 2-(3,5-dichlorophenyl-2,4,6-d₃)-1,3-benzoxazole-6-carboxylic-7-d acid-d, 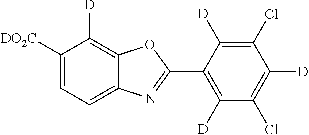
(d) 2-(3,5-dichlorophenyl-2,4,6-d₃)-1,3-benzoxazole-6-carboxylic-7-d acid, 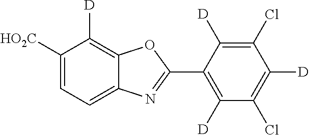 [SMILES: [2H]c1c(Cl)c([2H])c(-c2nc3ccc(C(=O)O)c([2H])c3o2)c([2H])c1Cl]
(e) 2-(3,5-dichlorophenyl-2,4,6-d₃)-1,3-benzoxazole-6-carboxylic-4,7-d₂ acid-d, 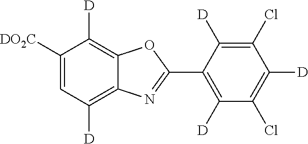
(f) 2-(3,5-dichlorophenyl-2,4,6-d₃)-1,3-benzoxazole-6-carboxylic-4,7-d₂ acid, 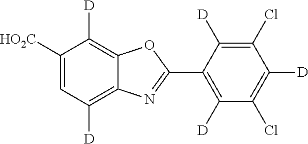 [SMILES: [2H]c1c(Cl)c([2H])c(-c2nc3c([2H])cc(C(=O)O)c([2H])c3o2)c([2H])c1Cl]
(g) 2-(3,5-dichlorophenyl-2,4,6-d₃)-1,3-benzoxazole-6-carboxylic-4,5,7-d₃ acid-d, 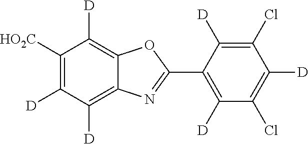 [SMILES: [2H]c1c(Cl)c([2H])c(-c2nc3c([2H])c([2H])c(C(=O)O)c([2H])c3o2)c([2H])c1Cl]
(h) 2-(3,5-dichlorophenyl-2,4,6-d₃)-1,3-benzoxazole-6-carboxylic-4,5,7-d₃ acid, 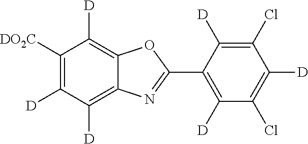
(j) 2-(3,5-dichlorophenyl-2,4,6-d₃)-1,3-benzoxazole-6-carboxylic-4-d₁ acid-d, 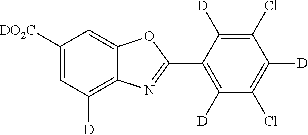
(k) 2-(3,5-dichlorophenyl-2,4,6-d₃)-1,3-benzoxazole-6-carboxylic-4-d acid, 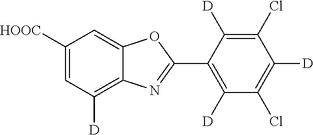 [SMILES: [2H]c1c(Cl)c([2H])c(-c2nc3c([2H])cc(C(=O)O)cc3o2)c([2H])c1Cl]
(l) 2-(3,5-dichlorophenyl-2,4,6-d₃)-1,3-benzoxazole-6-carboxylic-5,7-d₂ acid-d, 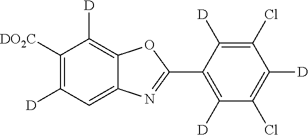
(m) 2-(3,5-dichlorophenyl-2,4,6-d₃)-1,3-benzoxazole-6-carboxylic-5,7-d₂ acid,

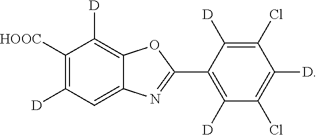

2. The compound of claim 1 selected from the group consisting of:
(a) 2-(3,5-dichlorophenyl-2,4,6-d₃)-1,3-benzoxazole-6-carboxylic acid, 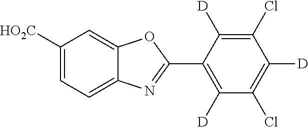 [SMILES: [2H]c1c(Cl)c([2H])c(-c2nc3ccc(C(=O)O)cc3o2)c([2H])c1Cl]
(b) 2-(3,5-dichlorophenyl-2,4,6-d₃)-1,3-benzoxazole-6-carboxylic-7-d acid, 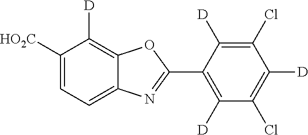 [SMILES: [2H]c1c(Cl)c([2H])c(-c2nc3ccc(C(=O)O)c([2H])c3o2)c([2H])c1Cl]
(c) 2-(3,5-dichlorophenyl-2,4,6-d₃)-1,3-benzoxazole-6-carboxylic-4,7-d₂ acid, 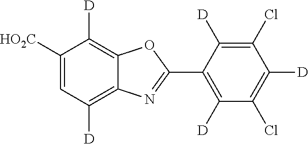 [SMILES: [2H]c1c(Cl)c([2H])c(-c2nc3c([2H])cc(C(=O)O)c([2H])c3o2)c([2H])c1Cl]
(d) 2-(3,5-dichlorophenyl-2,4,6-d₃)-1,3-benzoxazole-6-carboxylic-4,5,7-d₃ acid, 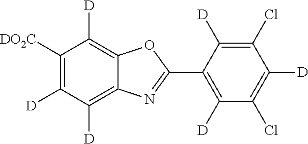
(e) 2-(3,5-dichlorophenyl-2,4,6-d₃)-1,3-benzoxazole-6-carboxylic-4-d acid, 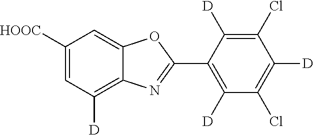 [SMILES: [2H]c1c(Cl)c([2H])c(-c2nc3c([2H])cc(C(=O)O)cc3o2)c([2H])c1Cl]
(f) 2-(3,5-dichlorophenyl-2,4,6-d₃)-1,3-benzoxazole-6-carboxylic-5,7-d₂ acid,

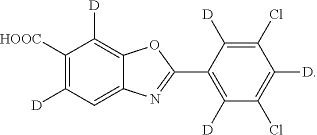

3. The compound of claim 2 that is
(2-(3,5-dichlorophenyl-2,4,6-d₃)-1,3-benzoxazole-6-carboxylic acid,

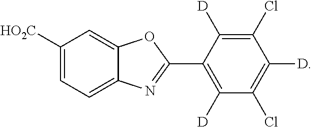

4. A pharmaceutical composition comprising a compound of claim 2 and a pharmaceutically acceptable carrier.

5. The pharmaceutical composition of claim 4 in an amount effective for the treatment of a disease selected from transthyretin amyloidosis cardiomyopathy (ATTR-CM), transthyretin amyloidosis polyneuropathy (ATTR-PN), neuropathy, senile systemic amyloidosis, cerebral amyloidosis, Alzheimer's disease, neuropathic amyloidosis, neuropathy, and gastrointestinal amyloidosis.

6. The pharmaceutical composition of claim 5 in an amount effective for the treatment of a disease selected from transthyretin amyloidosis cardiomyopathy (ATTR-CM), and transthyretin amyloidosis polyneuropathy (ATTR-PN).

7. A method of treating a disease selected from ATTR-CM, ATTR-PN, neuropathy, senile systemic amyloidosis, cerebral amyloidosis, neuropathic amyloidosis, neuropathy, and gastrointestinal amyloidosis, ocular amyloidosis, leptomeningeal amyloidosis, comprising administering a pharmaceutically effective amount of the pharmaceutical composition of claim 2.

8. The method of claim 7, wherein the disease is selected from the group consisting of familial amyloid cardiomyopathy (ATTR-CM), familial amyloid polyneuropathy (ATTR-PN), senile systemic amyloidosis, cerebral amyloidosis, neuropathic amyloidosis, neuropathy, and gastrointestinal amyloidosis, ocular amyloidosis, leptomeningeal amyloidosis, and with a therapeutically effective dose of the compound of claim 3.

9. The method of claim 8, wherein said transthyretin amyloid (ATTR) disease is familial amyloid cardiomyopathy (ATTR-CM).

10. The method of claim 8, wherein said transthyretin amyloid (ATTR) disease is familial amyloid polyneuropathy (ATTR-PN).

11. The method of claim 8, wherein said transthyretin amyloid (ATTR) disease is senile systemic amyloidosis.

12. The method of claim 8, wherein said transthyretin amyloid (ATTR) disease is ocular amyloidosis.

13. The method of claim 8, wherein said transthyretin amyloid (ATTR) disease is leptomeningeal amyloidosis.
